(19) United States
(12) Patent Application Publication
(10) Pub. No.: US 2004/0221330 A1
(43) Pub. Date: Nov. 4, 2004

(54) Processes and vectors for producing transgenic plants

(76) Inventors: Victor Klimyuk, Halle/Saale (DE); Gregor Benning, Halle/Saale (DE); Serik Eliby, Halle/Saale (DE); Yuri Gleba, Halle/Saale (DE)

Correspondence Address: MYERS BIGEL SIBLEY & SAJOVEC, PO BOX 37428, RALEIGH, NC, 27627, US

(21) Appl. No.: 10/479,706

(22) PCT Filed: Jun. 12, 2002
(86) PCT No.: PCT/EP02/06464

(30) Foreign Application Priority Data
Jun. 13, 2001 (DE) 10129010.1

(51) Int. Cl. C12N 15/82; C07H 21/02; C12N 15/87; C07K 14/00; C12N 5/04; C12N 15/90; C12N 5/10

(52) U.S. Cl. 800/278; 800/286; 800/288; 536/23.1; 530/350; 435/419; 435/468

(57) ABSTRACT

A process for producing transgenic plants or plant cells capable of expressing a coding sequence of interest under transcriptional and translational control of host nuclear transcriptional and translational elements is described by introducing into the nuclear genome of host plants or plant cells a vector comprising said coding sequence of interest which is devoid of (a) an upstream element of initiation of transcription functional in the host plants or plant cells and operably linked to said coding sequence of interest and required for its transcription; (b) an upstream element of initiation of translation functional in the host plants or plant cells and operably linked to said coding sequence of interest; and subsequently selecting plant cells or plants expressing said coding sequence of interest.

### FIELD OF THE INVENTION

The present invention relates to processes and vectors for producing transgenic plants as well as plants and plant cells obtained thereby.

### BACKGROUND OF THE INVENTION

Achievement of a desirable and stably inheritable pattern of transgene expression remains one of the major problems in plant biotechnology. The standard approach is to introduce a transgene as part of a fully independent transcription unit in a vector, where the transgene is under transcriptional control of a plant-specific heterologous or a homologous promoter and transcription termination sequences (for example, see U.S. Pat. No. 5,591,605; U.S. Pat. No. 5,977,441; WO 0053762 A2; U.S. Pat. No. 5,352,605, etc). However, after the integration into the genomic DNA, because of random insertion of exogenous DNA into plant genomic DNA, gene expression from such transcriptional vectors becomes affected by many different host factors. These factors make transgene expression unstable, unpredictable and often lead to transgene silencing in the progeny (Matzke & Matzke, 2000, Plant Mol Biol., 43, 401-415; S. B. Gelvin, 1998, Curr. Opin. Biotechnol., 9, 227-232; Vaucheret et al., 1998, Plant J., 16, 651-659). There are well-documented instances of transgene silencing in plants, which include the processes of transcriptional (TGS) and posttranscriptional gene silencing (PTGS). Recent findings reveal a close relationship between methylation and chromatin structure in TGS and involvement of RNA-dependent RNA-polymerase and a nuclease in PTGS (Meyer, P., 2000, Plant Mol. Biol, 43, 221-234; Ding, S. W., 2000, Curr. Opin. Biotechnol., 11, 152-156; lyer et al., Plant Mol. Biol., 2000, 43, 323-346). For example, in TGS, the promoter of the transgene can often undergo methylation at many integration sites with chromatin structure not favorable for stable transgene expression. As a result, practicing existing methods requires many independent transgenic plants to be produced and analyzed for several generations in order to find those with the desired stable expression pattern. Moreover, even such plants displaying a stable transgene expression pattern through the generations can become subsequently silenced under naturally occurring conditions such as a stress or a pathogen attack. Existing approaches aiming at improved expression control, such as use of scaffold attachment regions (Allen, G. C., 1996, Plant Cell, 8, 899-913; Clapham, D., 1995, J. Exp. Bot., 46, 655-662; Allen, G. C., 1993, Plant Cell, 1, 603-613) flanking the transcription unit, could potentially increase the independence and stability of transgene expression by decreasing the dependence from so-called “position effect variation” (Matzke & Matzke, 1998, Curr. Opin. Plant Biol., 1, 142-148; S. B. Gelvin, 1998, Curr. Opin. Biotechnol., C 227-232; WO 9844 139 A1; WO 006757 A1; EP 1 005 560 A1; AU 00,018,331 A1). However, they only provide a partial solution to the existing problem of designing plants with a required expression pattern of a transgene.

Gene silencing can be triggered as a plant defense mechanism by viruses infecting the plant (Ratcliff et al., 1997, Science, 276, 1558-1560; Al-Kaff et al., 1998, Science, 279, 2113-2115). In non-transgenic plants, such silencing is directed against the pathogen, but in transgenic plants it can also silence the transgene, especially when the transgene shares homology with a pathogen. This is a problem, especially if many different elements of viral origin are used in designing transcriptional vectors. An illustrative example is the recent publication by Al-Kaff and colleagues (Al-Kaff et al., 2000, Nature Biotech., 18, 995-999) who demonstrated that CaMV (cauliflower mosaic virus) infection of a transgenic plant can silence the BAR gene under the control of the CaMV-derived 35S promoter. It is worth mentioning that all transgenic plants released so far into the environment and cultured commercially were engineered using the 35S promoter as the transcription promoting signal.

During the last years, the set of cis-regulatory elements has significantly increased and presently includes tools for sophisticated spatial and temporal control of transgene expression. These include several transcriptional elements such as various promoters and transcription terminators as well as translational regulatory elements/enhancers of gene expression. In general, translation enhancers can be defined as cis-acting elements which, together with cellular trans-acting factors, promote the translation of the mRNA. Translation in eukaryotic cells is generally initiated by ribosome scanning from the 5′ end of the capped mRNA. However, initiation of translation may also occur by a mechanism which is independent of the cap structure. In this case, the ribosomes are directed to the translation start codon by internal ribosome entry site (IRES) elements. These elements, initially discovered in picornaviruses (Jackson & Kaminski, 1995, RNA, 1985-1000), have also been identified in other viral and cellular eucaryotic mRNAs. IRESes are cis-acting elements that, together with other cellular trans-acting factors, promote assembly of the ribosomal complex at the internal start codon of the mRNA. This feature of IRES elements has been exploited in vectors that allow for expression of two or more proteins from polycistronic transcription units in animal or insect cells. At present, they are widely used in bicistronic expression vectors for animal systems, in which the first gene is translated in a cap-dependent manner and the second one is under the control of an IRES element (Mountford & Smith, 1995, Trends Genet., 4, 179-184; Martines-Salas, 1999, Curr Opin Biotech., 19, 458-464). Usually the expression level of a gene under the control of an IRES varies significantly and is within a range of 6-100% compared to cap-dependent expression of the first one (Mizuguchi et al., 2000, Mol. Ther., 1, 376-382). These findings have important implications for the use of IRESs, for example for determining which gene shall be used as the first one in a bicistronic vector. The presence of an IRES in an expression vector confers selective translation not only under normal conditions, but also under conditions when cap-dependent translation is inhibited. This usually happens under stress conditions (viral infection, heat shock, growth arrest, etc.), normally because of the absence of necessary trans-acting factors (Johannes & Sarnow, 1998, RNA, 4, 1500-1513; Sonenberg & Gingras, 1998, Cur. Opin. Cell Biol., 10, 268-275).

Translation-based vectors recently attracted the attention of researchers working with animal cell systems. There is one report which describes the use of an IRES-Cre recombinase cassette for obtaining tissue-specific expression of cre recombinase in mice (Michael et al., 1999, Mech. Dev., 85, 35-47). In this work, a novel IRES-Cre cassette was introduced into the exon sequence of the EphA2 gene, encoding an Eph receptor of protein tyrosine kinase expressed early in development. This work is of specific interest as it is the first demonstration of the use of translational vectors for tissue-specific expression of a transgene in animal cells that relies on transcriptional control of the host DNA. Another important application of IRES elements is their use in vectors for insertional mutagenesis. In such vectors, the reporter or selectable marker gene is under the control of an IRES element and can only be expressed if it inserts within the transcribed region of a transcriptionally active gene (Zambrowich et al., 1998, Nature, 392, 608-611; Araki et al., 1999, Cell Mol. Biol., 45, 737-750). However, despite the progress made in the application of IRESs in animal systems, IRES elements from these systems are not functional in plant cells. Moreover, since site-directed or homologous recombination in plant cells is extremely rare and of no practical use, similar approaches with plant cells were not contemplated.

There are significantly less data on plant-specific IRES elements. Recently, however, several IRESs that are also active in plants were discovered in tobamovirus crTMV (a TMV virus infecting Cruciferae plants) (Ivanov et al., 1997, Virology, 232, 32-43; Skulachev et al., 1999, Virology, 263, 139-154; WO 98/54342) and there are indications of IRES translation control in other plant viruses (Hefferon et at., 1997, J. Gen Virol., 78, 3051-3059; Niepel & Gallie, 1999, J. Virol., 73, 9080-9088). IRES technology has a great potential for the use in transgenic plants and plant viral vectors providing a convenient alternative to existing vectors. Up to date, the only known application of plant IRES elements for stable nuclear transformation is connected with the use of IRESs to express a gene of interest in bicistronic constructs (WO 98/54342). The construct in question comprises, in 5′ to 3′ direction, a transcription promoter, the first gene linked to the said transcription promoter, an IRES element located 3′ to the first gene and the second gene located 3′ to the IRES element, i.e., it still contains a full set of transcription control elements. Recently, in our international patent application (PCT/EP01/14421) we described the use of IRES-based translational vectors devoid of transcriptional regulatory elements. Surprisingly, we found that vectors used as negative control and devoid of any transcriptional and translational regulatory elements, still yeild the frequency of transformation, which is high enough for practical applications, e.g. for producing transgenic plants, expressing trait of interest as translational fusion with endogenic protein.

It is the object of this invention is to provide a novel process for producing transgenic plants or plant cells which are capable of stable expression of a coding sequence of interest integrated into the genome and which are little susceptible to transgene silencing.

### GENERAL DESCRIPTION OF THE INVENTION

This invention provides a process of producing transgenic plants or plant cells capable of expressing a coding sequence of interest under transcriptional and translational control of host nuclear transcriptional and translational elements by introducing into the nuclear genome of host plants or plant cells for said transgenic plants or plant cells a vector comprising said coding sequence of interest which is devoid of

(a) an upstream element of initiation of transcription functional in the host plants or plant cells operably linked to said coding sequence of interest and required for its transcription;

(b) an upstream element of initiation of translation functional in the host plants or plant cells and operably linked to said coding sequence of interest; and subsequently selecting plant cells or plants expressing said coding sequence of interest.

This invention further provides, in a process of producing transgenic plants or plant cells capable of expressing a useful trait, a process of expressing a coding sequence of interest under transcriptional and translational control of host nuclear transcriptional and translational elements by introducing into the nuclear genome of host plants or plant cells for said transgenic plants or plant cells a vector comprising said coding sequence of interest which is devoid of

(a) an upstream element of initiation of transcription functional in the host plants or plant cells operably linked to said coding sequence of interest and required for its transcription;

(b) an upstream element of initiation of translation functional in the host plants or plant cells and operably linked to said coding sequence of interest; and subsequently selecting plant cells or plants expressing said coding sequence of interest.

During experimentation with translational vectors we have found a new method of genetic transformation of plants or plant cells. It is based on the use of vectors that carry a coding sequence of interest devoid of any functional transcription or translation initiation elements (functional elements (a) and (b)) operably linked to it and being functional in the ost plants or plant cells. The coding sequence may or may not have a functional element of termination of transcription operably linked to it. Preferably, it has a translation stop signal (stop codon). These vectors are termed _{“}translation fusion vectors”. Comparison of the transformation efficiency using the transcriptional-, IRES-based translational- and translational fusion vectors revealed a very surprising result. The number of transformants with translational fusion vectors, which were initially intended as negative control in transformation experiments, was only 2-10 times lower than that obtained with IRES-based translational vectors. This transformation efficiency is well within practically useful limits. For example, translational fusion vector pIC1451 (FIG. 3) resulted in a number of Brassica napus transformants, which was only two times lower, compared to IRES-based translational vector pIC1301 (FIG. 2). Translational vectors comprise a translation initiation element like an IRES upstream of a coding sequence of interest and rely on the transcription machinery of the host plant.

FIG. 3 shows an example of the simplest form of a translational fusion vector according to the invention. It contains a coding sequence of interest and is devoid of functional transcription and translation initiation elements operably linked to it. The vector may optionally have a transcription terminator (35S terminator in FIG. 3). One embodiment of the process of the invention using such a translational fusion vector is depicted in FIG. 1A: Transformation should lead to the incorporation of the vector into a coding part (an exon) of a transcriptionally active gene of the host plant. Upon transcription, a hybrid mRNA is formed which compriseses RNA derived from the nuclear DNA of said transgenic plant or plant cells and RNA derived from said coding sequence of interest, i.e. a hybrid mRNA. After RNA processing (e.g. intron splicing, capping, poly adenylation), translation results in a fusion protein having a portion of a native host protein as N-terminal part and the gene product of the coding sequence of interest as a C-terminal part. Preferably, translation stops after said coding sequence of interest due to a translation stop signal.

FIG. 1B depicts a more complex general embodiment, wherein the vector comprises a coding sequence of interest (transgene 1) devoid of the functional elements (a) and (b) and a further cistron joined thereto and downstream thereof. In this case, the coding sequence of interest (transgene 1) preferably does not have a functional transcription termination element which terminates transcription after transgene 1. Said further cistron(s) may be operably linked to transcriptional and/or translational elements like a promoter or an IRES element downstream of said coding sequence of interest and upstream of said further cistron. Moreover, said further cistron(s) preferably have a transcription termination signal downstream thereof. Preferably, said cistron(s) are under translational control of IRES element(s). In the case shown in FIG. 1B, transcription and translation leads to a fusion protein comprising the gene product of the coding sequence of interest. A further cistron (transgene 2) is translated under control of an IRES element.

If the translational fusion vector contains said coding sequence of interest as the only coding sequence or cistron, said coding sequence preferably codes for a selectable marker to allow for selection of transformants. If the vector contains one or more further cistrons downstream of said coding sequence, one of said cistrons may code for a selectable marker.

In another preferred embodiment, the coding sequence of interest (preferably encoding a selectable marker) in the translational fusion vector is followed by DNA sequences recognizable by site-specific recombinases (FIG. 1C). A transformant obtained in the process of the invention may then be used to integrate any gene of interest in a second transformation. Said gene of interest may preferably be under translational control of an IRES element. The IRES element may be provided upstream of said sequence recognisable by a site-specific recombinase in the translational fusion vector. A transformant with a known and desired or suitable expression pattern may be chosen for said second transformation. Alternatively, the selectable marker gene in a transformant may be replaced by any gene of interest using sites for site-specific recombination in the translational fusion vector (see e.g. that shown in FIG. 4). Thus, the transgenic plants or plant cells produced by the process of the invention may be used for further genetic engineering, particularly for targeted transformation using site-specific recombination.

If the translational fusion vector contains further cistrons downstream of said coding sequence of interest, the transformation marker is preferably used as the first cistron in the vector. This preferred process has all advantages of IRES-based translational vectors, but may further increase the chance of transformant recovery. Such a direct selection for translation fusion-based expression allows also to directly select for other useful traits, such as, but not limited to, herbicide resistance.

The vectors for the process of this invention can easily be improved for example by incorporating splicing sites in order to increase the chance of “in-frame” fusions, thus significantly increasing the transformation efficiency.

Typically, the process of the invention leads to the formation of hybrid messenger RNA (mRNA) comprising RNA derived from nuclear DNA of said transgenic plants or plant cells and RNA derived from said coding sequence of interest. In a typical embodiment, said hybrid mRNA encodes a fusion protein. Said hybrid mRNA may also encode multiple heterologous polypeptide sequences, e.g. when said vector further contains one of more cistrons downstream of said coding sequence of interest. In a further embodiment, said hybrid mRNA contains a sequence which is at least partially complementary (anti-sense) to an mRNA native to said plant or plant cells for suppressing expression of said mRNA native to said plant or plant cells, e.g. for functional genomics analysis. In order to facilitate the inclusion of translational fusion vector into the hybrid mRNA, the trait encoding sequence of said vector can be preceeded by splice acceptor sites (FIGS. 6 and 7).

It is known that many proteins including those encoding the plant reporter GUS (Kertlundit et al., 1991, Proc. Natl. Acad. Sci. USA, 88, 5212-5216), GFP (Santa Cruz et al., 1996, Proc. Natl. Acad. Sci. USA, 93, 6286-6290) and transformation selectable markers NPTII (Vergunst et al., 1998, Nucleic Acids Res., 26, 2729-2734), APH(3′)II (Koncz et at., 1989, Proc. Natl. Acad. Sci. USA, 86, 8467-8471), BAR (Botterman et al., 1991, Gene, 102, 33-37) can preserve their activity as (translational) fusion proteins. However, this finding had a limited application, which did not go beyond, for example, gene trapping in plants (Koncz et al., 1989, Proc. Natl. Acad. Sci. USA, 86, 8467-8471; Sundaresan et al., 1995, Genes Dev., 9, 1797-1810) or studying protein localization/expression patterns. In all cases mentioned above, vectors with some sort of transcription and/or translation termination signals were used. Here, we demonstrate for the first time, that transformation markers can efficiently be used for directly selecting transformed plant cells as translational fusion products with resident gene-encoded proteins.

Further, the vector for the process of the invention may contain one or more sequences encoding proteolytic cleavage sites next to or within said coding sequence of interest or said cistrons downstream thereof. This allows to obtain the protein encoded by said coding sequence of interest cleaved from the primary expressed fusion protein. Said proteolytic cleavage site may be autocatalytic allowing self-cleavage of the fusion protein. Alternatively, cleavage of the expressed fusion protein may require a site-specific protease. Such a protease may be native to said plant or plant cells. Alternatively, the plant or plant cells may be genetically modified or transfected so as to provide a heterologous site-specific protease for cleavage of the fusion protein.

The process of the invention may be used for the production of transgenic plants, preferably transgenic crop plants. These plants preferably express a useful trait. Said trait may at least partially be the result of expression of said coding sequence of interest to give an RNA molecule, e.g. a ribosomal, a transfer or a messenger RNA (e.g. for antisense technology). Preferably, said trait is the result of expression of said coding sequence to give a polypeptide or protein. Further, said trait may be the result of expression of said coding sequence of interest and of one or more additional cistrons.

The processes of the invention have the advantage that the transgenic plants or plant cells produced contain a minimal number of xenogenetic elements, which makes transgene expression more stable and transgene silencing less likely. Preferably, the sequences and elements used in the vectors for said process are of plant origin further reducing the content of foreign sequences in the transgenic plants and plants cells produced.

### BRIEF DESCRIPTION OF THE DRAWINGS

FIG. 1 shows three of many possible translational fusion vector variants.

A—the simplest version of a translational fusion vector having a coding sequences of interest (transgene);

B—the vector contains a second transgene separated from the first one by an IRES element;

C—the vector contains an IRES and a recombination site (RS) recognized by a site-specific recombinase;

FIG. 2 depicts translational vector pIC1301 containing IRES_{MP,75}^{CR}, BAR and the 35S terminator.

FIG. 3 depicts vector pIC1451 containing a promoterless BAR gene and the 35S terminator.

FIG. 4 depicts vector pIC052 containing a loxP site, the HPT gene and a nos terminator.

FIG. 5 depicts vector pIC-BG containing the BAR-GFP translational fusion.

FIG. 6 depicts binary vector pICH3781, containing promoterless BAR gene preceded by three splice acceptor sites (3×SA).

FIG. 7 depicts binary vector pICH3831, containing promoterless BAR gene preceded by three splice acceptor sites (3×SA).

FIG. 8 depicts binary vector pICBV10.

### DETAILED DESCRIPTION OF THE INVENTION

Construction of vectors for stable transformation of plants has been described by numerous authors (for review, see Hansen & Wright, 1999, Trends in Plant Science, 4, 226-231; Gelvin, S. B., 1998, Curr. Opin. Biotech., Q 227-232). The basic principle of all these constructs is identical—a fully functional transcription unit consisting of, in 5′ to 3′direction, a plant-specific promoter, a structural part of a gene of interest and a transcriptional terminator, has to be introduced into the plant cell and stably integrated into the genome in order to achieve expression of a gene of interest.

We have developed a different technology for obtaining stable nuclear transformants of plants. Our invention relies on the surprising finding that introduction into a plant cell of coding sequences devoid of any functional transcription or translation initiation elements results in a relatively high frequency of transformants that express the coding sequence of interest, apparently as a result of the plant host's transcription/translation machinery being able to drive the formation of mRNA from a transgene of interest in a transformed plant cell. The proposed process utilizes vectors having a coding sequence of interest that is not operationally linked to a promoter or an IRES element in said vector, but, upon insertion into a coding part of the host genome, forms a translational fusion with a plant-encoded resident protein.

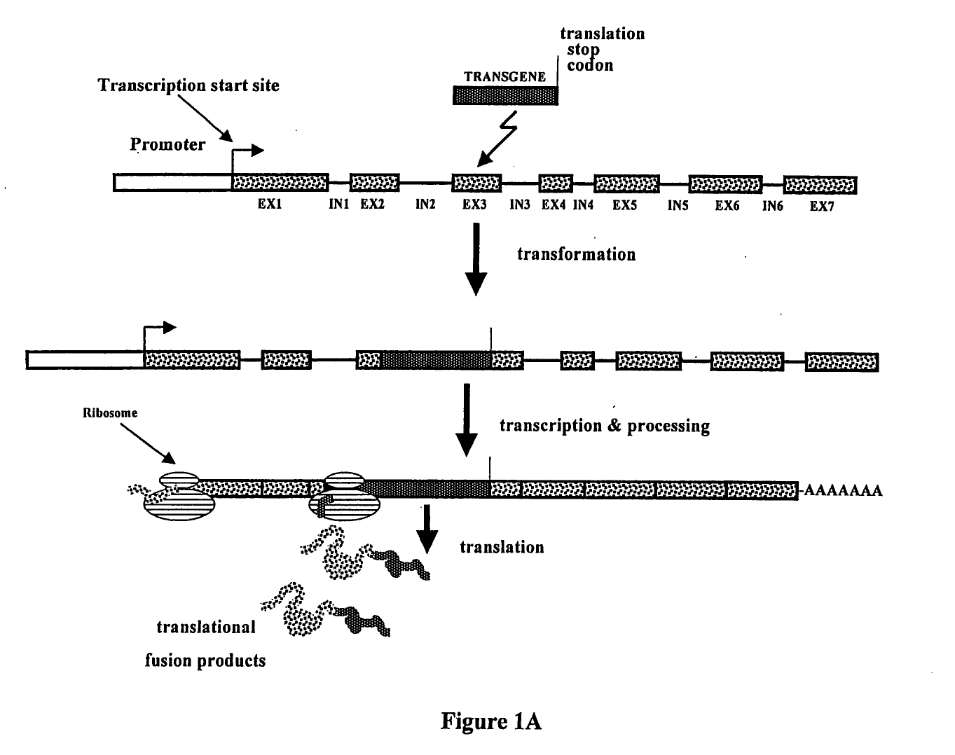

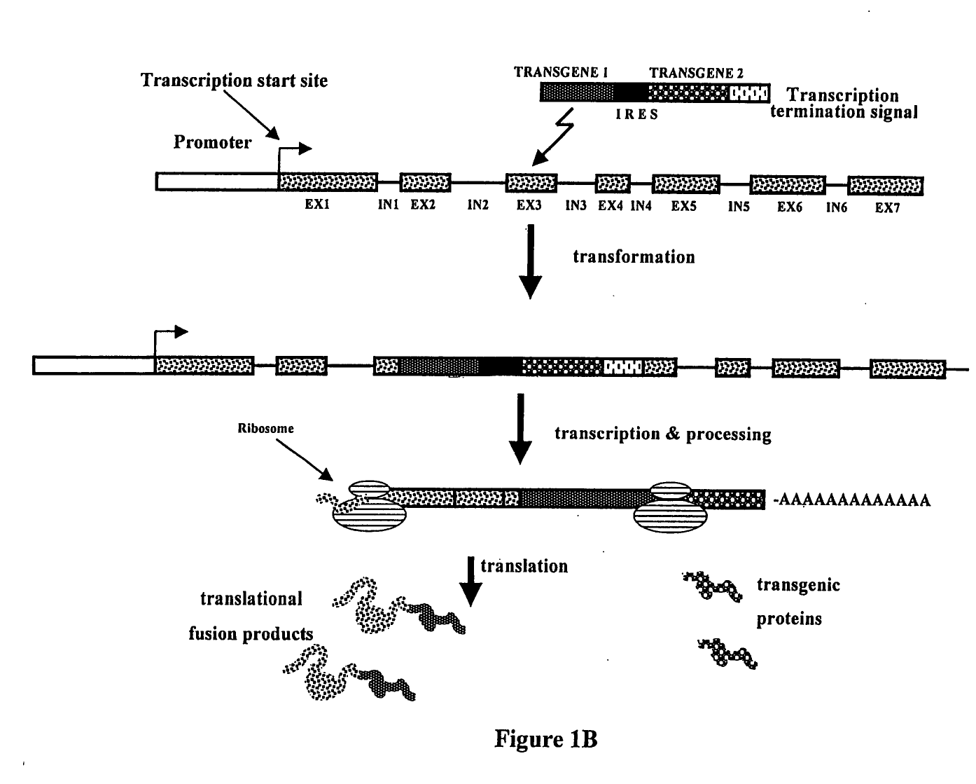

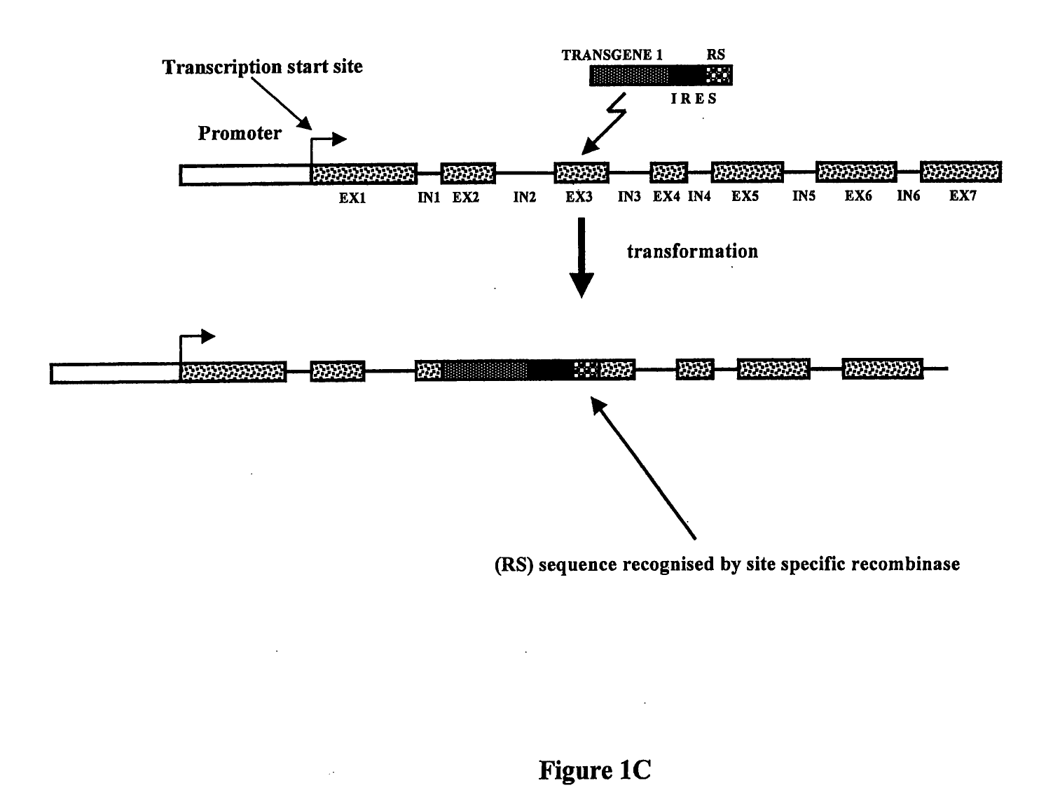

The vectors used in the process of the invention, after integration into the transcribed region of a resident plant gene, yield chimaeric mRNA which is subsequently translated into the fusion protein of interest (FIG. 1). To the best of our knowledge, there is no prior art concerning this approach for generating stable nuclear plant transformants. It was very surprising, that, given the low proportion of transcriptionally active DNA in most plant genomes, transformation experiments utilizing translation fusion vectors as described in the present invention, yield numerous transformants expressing the gene of interest.

This invention addresses imminent problems of reliable transgene expression. The transgene integrated into the host genome using the process of the invention, relies on the transcription/translation machinery including all or most of the transcriptional regulatory elements of the host's resident gene, thus minimizing transgene silencing usually triggered by xenogenetic regulatory DNA elements.

The vectors for transgene delivery can be built in many different ways. The simplest version consists of the coding sequence of a gene of interest or a portion thereof (basic translation fusion vector—FIG. 1A) and a transcription and a translation stop signal if desired. In another version, an IRES or a promoter element is incorporated after the coding sequence of interest to drive the transcription and/or translation of any additional cistrons. Advanced versions of the translational fusion vector may include sequences for site-specific recombination (for review, see Corman & Bullock, 2000, Curr Opin Biotechnol., 11, 455-460) allowing either the replacement of an existing transgene or integration of any additional gene of interest into the transcribed region of the host DNA (FIG. 1C). Site-specific recombinases/integrases from bacteriophages and yeasts are widely used for manipulating DNA In vitro and in plants. Examples for recombinases-recombination sites for the use in this invention include the following: cre recombinase-LoxP recombination site, FLP recombinase-FRT recombination sites, R recombinase-RS recombination sites, phiC31 integrase-attPlattB recombination sites etc.

The introduction of splicing sites into the translation vector may be used to increase the probability of transgene incorporation into the processed transcript.

The vector may further comprise a sequence coding for a targeting signal peptide upstream of said coding sequence of interest or said additional cistron(s). Preferable examples of such signal peptides include a plastid transit peptide, a mitochondrial transit peptide, a nuclear targeting signal peptide, a vacuole targeting peptide, and a secretion signal peptide.

Vectors that include proteolytic sites flanking the coding sequence of interest will result in cleavage of the fusion protein and release of the protein of interest in a pure form, if the conditions are provided that allow for such proteolytic cleavage.

Various methods can be used to deliver translational vectors into plant cells, including direct introduction of said vector into a plant cell by means of microprojectile bombardment, electroporation or PEG-mediated treatment of protoplasts. Agrobacterium-mediated plant transformation also presents an efficient way of the translational vector delivery. The T-DNA insertional mutagenesis in Arabidopsis and Nicotiana with the promoterless reporter APH(3′)II gene closely linked to the right T-DNA border showed that at least 39% of all inserts induced transcriptional and translational gene fusions (Koncz et al., 1989, Proc. Natl. Acad. Sci., 86, 8467-8471).

All approaches described above aim at designing a system that places a coding sequence of interest under expression control of a resident gene in which the insertion occurred. This may result in a suitable expression level of sequence of interest. In many other cases, a modified pattern of transgene expression may be preferred. In these cases, the translation fusion vector can be equipped with transcriptionally active elements such as enhancers which can modulate the expression pattern of a transgene. It is known that enhancer sequences can affect the strength of promoters located as far as several thousand base pairs away (Müller, J., 2000, Current Biology, 10 R241-R244). The feasibility of such an approach was demonstrated in experiments with activation tagging in Arabidopsis (Weigel et al., 2000, Plant Physiol., 122, 1003-1013), where T-DNA-located 35S enhancer elements changed the expression pattern of resident genes, and in enhancer-trap transposon tagging described above. In the latter example, resident gene enhancers determined the expression pattern of the reporter transgene. This approach might be useful, for example, at the initial stages of plant transformation, or when modulation of the transgene expression pattern is required after the transformation.

The expression pattern may also be modulated by using translational enhancers. The enhancer sequences can be easily manipulated by means of sequence-specific recombination systems (inserted, replaced or removed) depending on the needs of the application. However, enhancers cannot function as initiators of transcription or translation.

Our approach was to preferably make a set of constructs based on a plant selectable marker gene functional as translational fusion protein. Such a marker gene can be preceded or followed by a recombination site recognized by site-specific recombinase, thus allowing the integration of any gene of interest at a predetermined site, by employing an additional transformation step. Optionally, the marker gene can be followed by another transgene (cistron) under the control of an IRES or a promoter. These constructs can be used directly for plant cell transformation after being linearized from the 5′ end in front of the coding sequence of interest or can be cloned into the T-DNA for Agrobacterium-mediated DNA transfer.

The further set of constructs aims at expressing a desirable trait as a stand-alone fusion product. In these experiments, a coding sequence of interest has to confer a selection advantage, such as, but not limited to, herbicide resistance. Our example is built on the use of a translation fusion vector to create a plant expressing resistance to the Basta herbicide, by having a fusion protein that contains a functional part of the enzyme.

This approach can be used also if the sequence of interest is an antisense sequence and the transcription results in creation of hybrid RNA, a part of which is antisense designed to silence an endogenous gene.

Another set of constructs, serving as controls, may contain either a promoterless selectable gene under IRES control, (a positive translational vector) or a selectable gene under the control of a constitutive promoter functional in monocot and/or dicot cells (a positive control or transcriptional vector). DNA was transformed into plant cells using different suitable technologies, such as Ti-plasmid vector carried by Agrobacterium (U.S. Pat. No. 5,591,616; U.S. Pat. No. 4,940,838; U.S. Pat. No. 5,464,763), particle or microprojectile bombardment (U.S. Pat. No. 5,100,792; EP 00444882 B1; EP 00434616 B1). In principle, other plant transformation methods could be used, such as but not limited to, microinjection (WO 09209696; WO 09400583 A1; EP 175966 B1), electroporation (EP 00564595 B1; EP 00290395 B1; WO 08706614A1).

The transformation method depends on the plant species to be transformed. Our exemplification includes data on the transformation efficiency for representatives of monocot (e.g. Triticum monococcum) and dicot (e.g. Brassica napus, Orichophragmus violaceous) plant species, thus demonstrating the feasibility of our approach for plant species of different phylogenetic origin and with different densities of transcribed regions within a species genome.

The transgenic coding sequence in the vector may represent only part of a gene of interest, which gene is then reconstructed to a functional length as a result of subsequent site-directed or homologous recombination.

### EXAMPLES

### Example 1

Construction of IRES-Containing and Translational Fusion Vectors

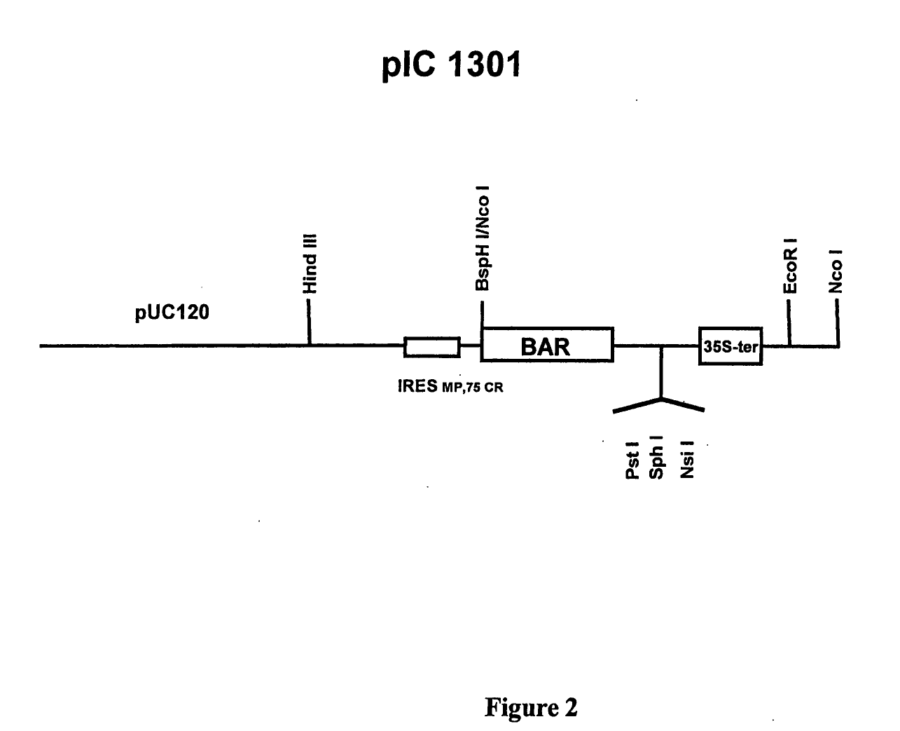

Series of IRES-mediated expression vectors were constructed using standard molecular biology techniques (Maniatis et al., 1982, Molecular cloning: a Laboratory Manual. Cold Spring Harbor Laboratory, New York). Vector pIC1301 (FIG. 2) was made by digesting plasmid pIC501 (p35S-GFP-IRES_{MP,75}^{CR}-BAR-35S terminator in pUC120) with HindIII and religating large gel-purified fragment. The IRES_{MP,75}^{CR} sequence represents the 3′ terminal 75 bases of the 5′-nontranslated leader sequence of the subgenomic RNA of the movement protein (MP) of a crucifer (CR)-infecting tobamovirus.

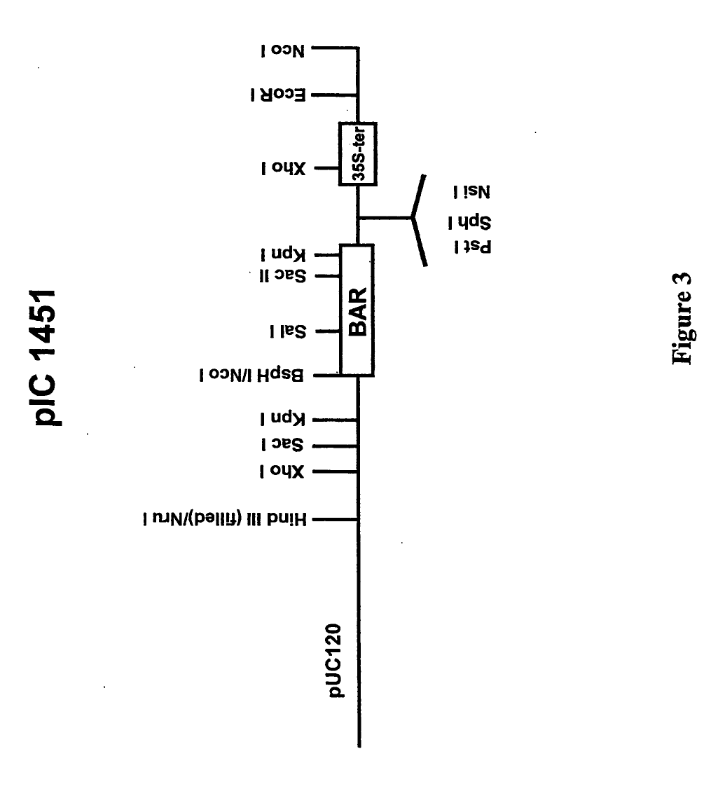

A construct containing a promoterless BAR gene was made by deleting the 35S promoter from a plasmid containing p35S:BAR-3′35S (pIC1311, not shown). Plasmid pIC1311 was digested with HindIII-NruI and blunt-ended by treatment with Klenow fragment of DNA polymerase 1. The large restriction fragment was gel-purified and religated producing pIC1451 (promoterless BAR-35S terminator; see FIG. 3).

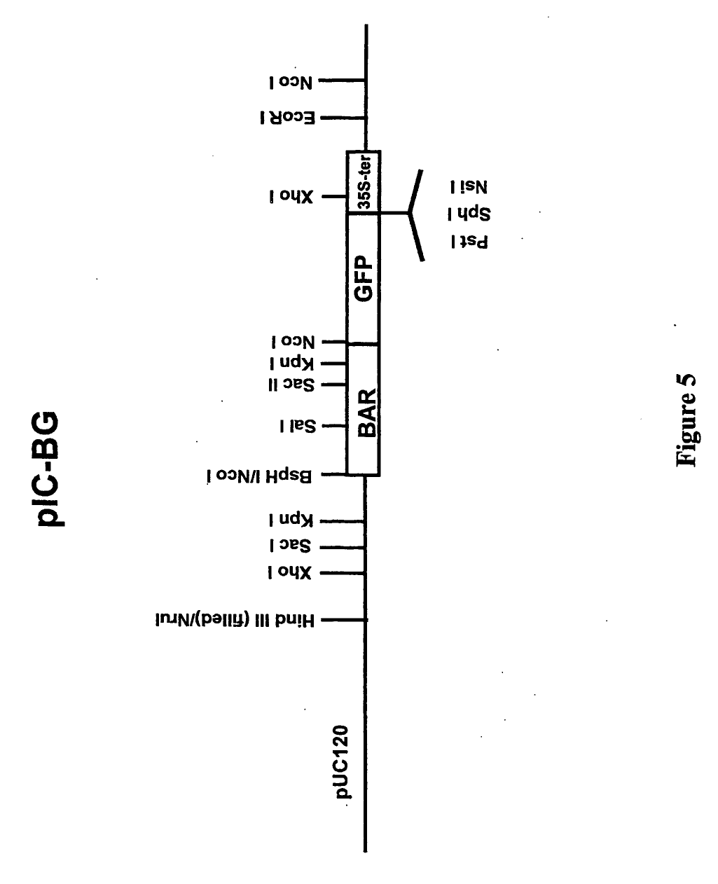

The vector pIC-BG (FIG. 5) was made as follows: the 3′-end of the BAR-gene was PCR-amplified using plasmid pIC026 as template and two BAR-gene-specific primers (forward primer: 5′-acgcgtcgaccgtgtacgtctccc-3′ and reverse primer: 5′-ccatggcgatctcggtgacgggc aggac-3′). With these primers, a Sal I- and a Nco I-site were introduced at the 5′- and 3′-end of this PCR-fragment, respectively. To clone the final BAR/GFP-fusion construct, this Sal I/Nco I digested and gel-purified PCR-product was ligated with the gel-purified small Nco I/Pst I-fragment of construct pIC011 (HBT promoter: GFP-NOS term) and the gel-purified large fragment of construct pIC1451 was digested with Sal I and Pst I. In this construct (pIC BG) the bar gene is fused in frame to the 5′-end of the GFP-gene. On the protein level, a BAR-GFP-fusion protein can be expressed from this construct, wherein the BAR-protein part is separated by one amino acid (Ala) from the GFP-protein. The amplified part of this construct was sequenced to confirm the sequence.

All vectors were linearized for use in the transformation experiments by digesting either with SacI (pIC1451, pIC BG) or HindIII (pICO52; pIC1301) restriction enzyme and gel-purified to separate from undigested vectors.

### Example 2

PEG-Mediated Orotoplast Transformation of Brassica napus

Isolation of Protoplasts

The isolation of Brassica protoplasts was based on previously described protocols (Glimelius K., 1984, Physiol.Plant., 61, 3844; Sundberg & Glimelius, 1986, Plant Science, 43, 155-162 and Sundberg et al., 1987, Theor. Appl. Genet., 75, 96-104).

Sterilized seeds (see Appendix) were germinated in 90 mm Petri dishes containing 1/2 MS medium with 0.3% Gelrite. The seeds were placed in rows slightly separated from each other. The Petri dishes were sealed, tilted at an angle of 45° and kept in the dark for 6 days at 28° C. The hypocotyls were cut into 1-3 mm long peaces with a sharp razor blade. The blades were often replaced to avoid the maceration of the material. The peaces of hypocotyls were placed into the TVL solution (see Appendix) to plasmolise the cells. The material was treated for 1-3 hours at room temperature. This pre-treatment significantly improves the yield of intact protoplasts. The preplasmolysis solution was replaced with 8-10 ml of enzyme solution (see Appendix). The enzyme solution should cover all the material but should not to be used in excess. The material was incubated at 20-25° C. in the dark for at least 15 hours. The Petri dishes were kept on a rotary shaker with very gentle agitation. The mixture of protoplasts and cellular debris was filtered through 70 mm mesh size filter. The Petri dishes were rinsed with 5-10 ml of W5 solution (Menczel et al., 1981, Theor. Appl. Genet., 59, 191-195) (also see Appendix) that was also filtered and combined with the rest of the suspension. The protoplast suspension was transferred to 40 ml sterile Falcon tubes and the protoplasts were pelleted by centrifugation at 120 g for 7 min. The supernatant was removed and the pellet of protoplasts was re-suspended in 0.5 M sucrose. The suspension was placed into 10 ml sterile centrifuge tubes (8 ml per tube) and loaded with 2 ml of W5 solution. After 10 min of centrifugation at 190 g the intact protoplasts were collected from the interphase with a Pasteur pipette. They were transferred to new centrifuge tubes, resuspended in 0.5 M mannitol with 10 mM CaCl₂ and pelleted at 120 g for 5 min.

PEG Treatment

The protoplasts were resuspended in the transformation buffer (see Appendix). The protoplast concentration was determined using the counting chamber and then adjusted to 1 -1.5×10⁶ protoplasts/ml. A 100 μl drop of this suspension was placed at the lower edge of the tilted 6-cm Petri dish and left for a few minutes allowing the protoplasts to settle. The protoplasts were then gently mixed with 50-100 μl of DNA solution (Qiagen purified, dissolved in TE at the concentration 1 mg/ml). Then 200 μl of PEG solution (see Appendix) was added dropwise to the protoplast/DNA mixture. After 15-30 min the transformation buffer (or W5 solution) was added in small aliquots (dropwise) until the dish was almost filled (6 ml). The suspension was left to settle for 1-5 hours. Then the protoplasts were transferred to centrifuge tubes, re-suspended in W5 solution and pelleted at 120 g for 5-7 min.

Protoplast Culture and Selection for Transformants

The protoplasts were transferred to the culture media 8 μM (Kao & Michayluk, 1975, Planta, 126, 105-110; also see the Appendix) and incubated at 25° C., low light density, in 2.5 cm or 5 cm Petri dishes with 0.5 ml or 1.5 ml of media, respectively. Protoplast density was 2.5×10⁴ protoplasts/ml. The three volumes of fresh 8 μM media without any hormones were added right after the first protoplasts division. The cells were incubated at high light intensity, 16 hours per day.

After 10-14 days, the cells were transferred to K3 media (Nagy & Maliga, 1976, Z. Pflanzenphysiol., 78, 453-455) with 0.1 M sucrose, 0.13% agarose, 5-15 mg/L of PPT and the hormone concentration four times less than in the 8 μM medium. To facilitate the transfer to fresh media, the cells were placed on the top of sterile filter paper by carefully spreading them in a thin layer. The cells were kept at high light intensity, 16 hours per day. The cell colonies were transferred to Petri dishes with differentiation media K3 after their size had reached about 0.5 cm in diameter.

### Example 3

Transformation of Triticum monococcum by Microprojectile Bombardment

Plant Cell Culture

Suspension cell line of T. monococcum L. was grown in MS2 (MS salts (Murashige & Skoog, 1962 Physiol. Plant., 15, 473-497), 0.5 mg/L Thiamine HCl, 100 mg/L inosit, 30 g/L sucrose, 200 mg/L Bacto-Tryptone, 2 mg/L 2,4-D) medium in 250 ml flasks on a gyrotary shaker at 160 rpm at 25° C. and was subcultured weekly. Four days after a subculture, the cells were spread onto sterile 50 mm filter paper disks on a gelrite-solidified (4 g/L) MS2 with 0.5 M sucrose.

Microprojectile Bombardment

Microprojectile bombardment was performed utilizing the Biolistic PDS-1000/He Particle Delivery System (Bio-Rad). The cells were bombarded at 900-1100 psi, at 15 mm distance from a macrocarrier launch point to the stopping screen and 60 mm distance from the stopping screen to a target tissue. The distance between the rupture disk and the launch point of the macrocarrier was 12 mm. The cells were bombarded after 4 hours of osmotic pretreatment.

A DNA-gold coating according to the original Bio-Rad's protocol (Sanford et al., 1993, In: Methods in Enzymology, ed. R.Wu, 217, 483-509) was done as follows: 25 μl of gold powder (0.6, 1.0 mm) in 50% glycerol (60 mg/ml) was mixed with 5 μl of plasmid DNA at 0.2 μg/μl, 25 μl CaCl₂ (2.5 M) and 10 μl of 0.1 M spermidine. The mixture was vortexed for 2 min followed by incubation for 30 min at room temperature, centrifugation (2000 rpm, 1 min), washing by 70% and 99.5% ethanol. Finally, the pellet was resuspended in 30 μl of 99.5% ethanol (6 μl/shot).

A new DNA-gold coating procedure (PEG/Mg) was performed as follows: 25 μl of gold suspension (60 mg/ml in 50% glycerol) was mixed with 5 μl of plasmid DNA in an Eppendorf tube and supplemented subsequently by 30 μl of 40% PEG in 1.0 M MgCl₂. The mixture was vortexed for 2 min and than incubated for 30 min at room temperature without mixing. After centrifugation (2000 rpm, 1 min) the pellet was washed twice with 1 ml of 70% ethanol, once by 1 ml of 99.5% ethanol and dispersed finally in 30 μl of 99.5% ethanol. Aliquots (6 μl) of DNA-gold suspension in ethanol were loaded onto macrocarrier disks and allowed to dry up for 5-10 min.

Plasmid DNA Preparation

Plasmids were transformed into E. coli strain DH10B, maxi preps were grown in LB medium and DNA was purified using the Qiagen kit.

Selection

For stable transformation experiments, the filters with the treated cells were transferred onto the solid MS2 medium with the appropriate filter-sterilized selective agent (150 mg/L hygromycin B (Duchefa); 10 mg/L bialaphos (Duchefa). The plates were incubated in the dark at 26° C.

### Example 4

Transformation of Orychophragmus violaceus by Microprojectile Bombardment

Preparation of the Suspension Culture

Plants of O. violaceus are grown in vitro on MS medium, 0.3% Gelrite (alternatively, ½ MS, 2% sucrose and 0.8% agar) at 24° C. and 16/8 hours day/night photoperiod for 3-4 weeks. Four to six leaves (depending on their size) were cut into small peaces and transferred to the Magenta box with 30 ml of Callus Inducing Medium (CIM) (see Appendix). The material was kept for 4-5 weeks at dim light (or in dark) at 24° C. and vigorous agitation. During this period the fresh CIM media was added to keep the plant tissue in the Magenta box covered with liquid. The cells sticking to the wall of the Magenta box were released into the media by vigorous inverting and shaking of the box.

Preparation of Plant Material for Microprojectile Bombardment

An aliquote of cell suspension was carefully placed onto the sterile filter paper supported by solid CIM media in a Petri dish. The Petri dish with plant material was kept in the dark for 5-7 days. Four hours before the procedure, the filter paper with cells was moved to fresh CIM with 10% sucrose. Microprojectile bombardment was performed as described in Example 3. Fourteen hours after the bombardment the material was transferred to CIM with 3% sucrose and kept in the dark.

Selection for Transformants

Two to four days after the bombardment, the filter paper with cells was transferred to the plate with CIM supplemented with the appropriate selection agent (10-15 μg/ml PPT). Every seven days the material was transferred to fresh selection media. The plates were kept in the dark and after approximately 6 weeks the plant material was transferred to the Petri plates with Morphogenesis Inducing Medium (MIM) (see Appendix) supplemented with the appropriate selection agent (10-15 μg/ml PPT). The plates were incubated at high light intensity, 16 hours day length.

### Example 6

Transformation of Triticum monococcum with Promoterless loxP-HPT Gene

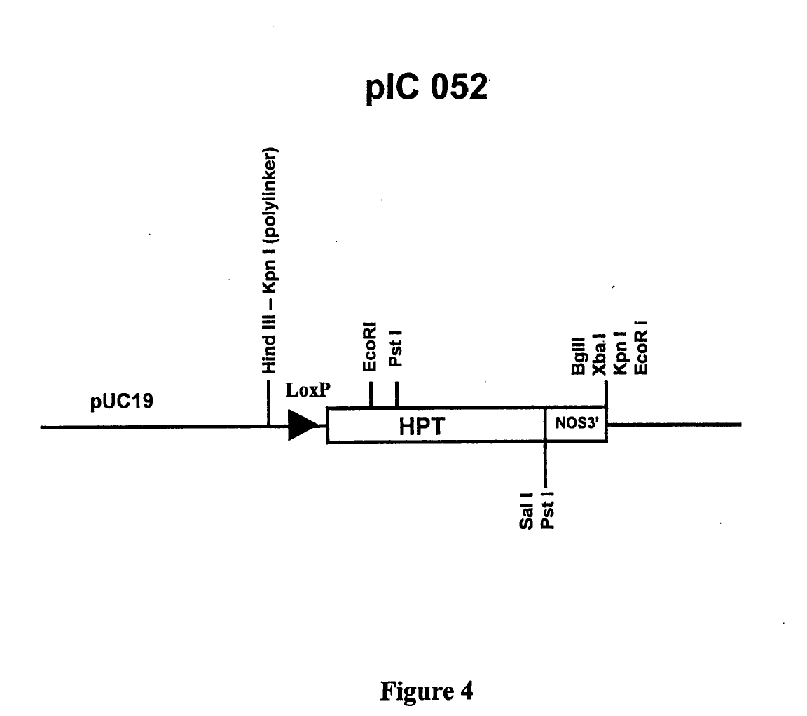

The construct pIC052 (FIG. 4) was linearized by digestion with HindIII restriction enzyme, gel-purified to separate undigested material and used for the microprojectile bombardment as described above (see EXAMPLE 3). The linearized vector contains pUC19 polylinker (57 bp) followed by a loxP site from the 5′ end of the HPT gene. In general, approximately 100 bp is located at the 5′ end of the translation start codon of the HPT gene. Thirty four plates were transformed and after 1.5 months of selection on hygromycin-containing media (EXAMPLE 3), three hygromycin resistant colonies were recovered. The sequence of the integration sites recovered by PCR, confirmed the independency of all three transformants.

### Example 6

T-DNA Based Translational Fusion Vectors

The aim of this example is to demonstrate an Agrobacterium-mediated delivery of translational vectors into plant cells.

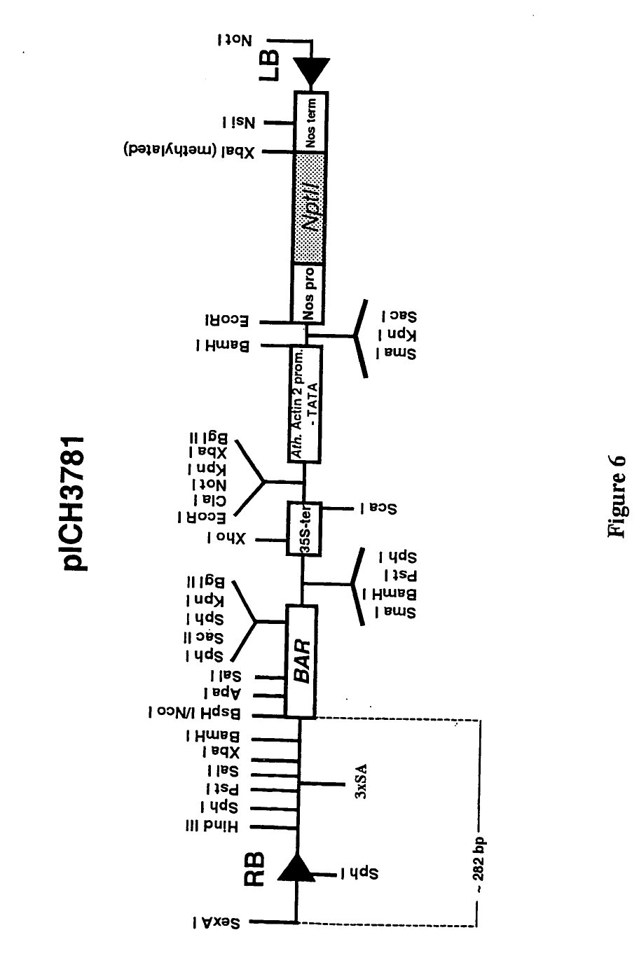

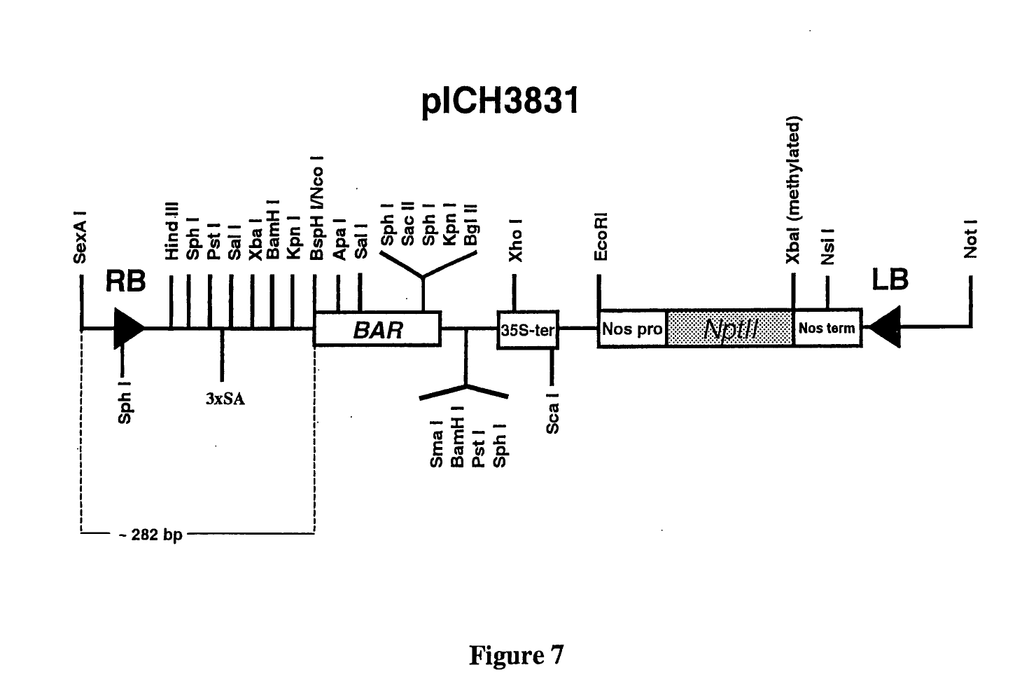

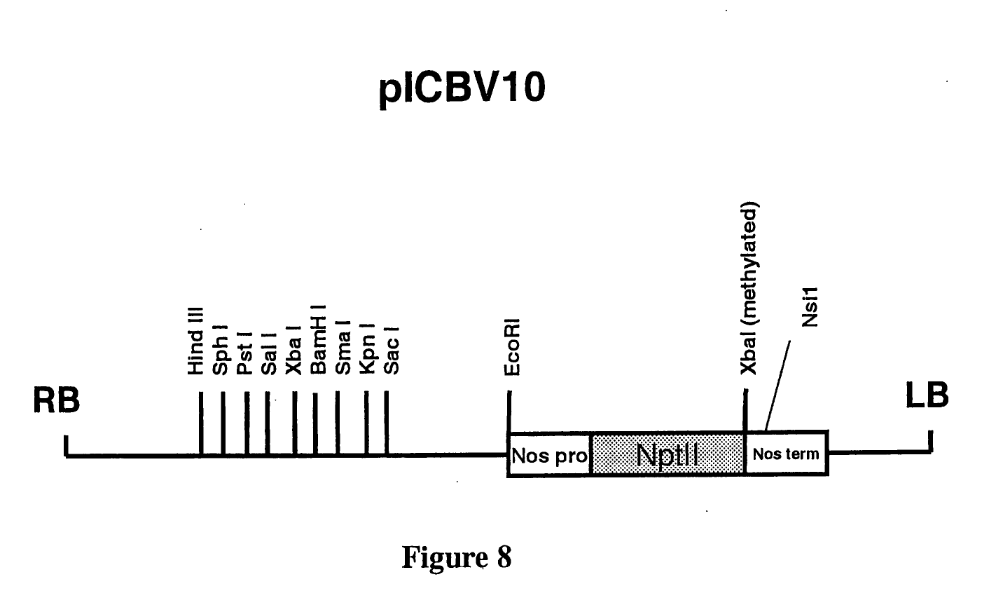

Further improvement of existing translational fusion vectors was achieved by subcloning of different vector elements into the binary vector pICBV10 (see FIG. 8) to enable the Agrobacterium tumefaciens mediated transformation of dicot plants. Both binary vectors were constructed using standard molecular biology techniques (Maniatis et al., 1982, Molecular cloning: a Laboratory Manual, Cold Spring Harbor Laboratory, New York). To construct vector pICH3781 (see FIG. 6) the promoterless expression cassette of construct pICH3651 (BAR-gene/terminator/enhancer element) was subcloned in a three fragment ligation as XbaI/EcoRI- and EcoRI/BamHI-fragment into the polylinker of pICBV10. Construct pICH3831 represents the same translation fusion vector like vector pICH3871 without the enhancer element (Actin 2-promoter without TATA-box, see FIG. 7). In order to remove this enhancer element, construct pICH3781 was EcoRI-digested and religated. Both construct pICH3781 and pICH3831 contain BAR gene preceded by three splice acceptor sites (SA) in order to facilitate the incorporation of BAR coding sequence into the processed transcript of residential gene and formation of correct translational fusion product.

In order to compare the efficiency of translational versus transcriptional vectors, the NPTII gene under control of NOS promoter was also incorporated into pICH3781 and pICH3831. The T-DNA of pICH3781 and pICH3831 were introduced in Arabidopsis thaliana (Col-0) plants as descried by Bent et al., (1994, Science, 285, 1856-1860). Seeds were harvested three weeks after vacuum-infiltration and divided in two equal groups. One group was sterilised and screened for transformants on GM+1% glucose medium (Valvekens et al., 1988, Proc. Natl. Acad. Sci. USA, 85, 5536-5540.) containing 50 mg L⁻¹ kanamycin. The other group was germinated in soil and sprayed several times by phosphinothricin solution (50 μg/ml). The number of transformants from each screening experiment was counted. The ratio of the number of transformants obtained with translational vectors to that obtained with transcriptional vectors (ppt^{R}:Km^{R}) was roughly in the range of 1:15-1:25 depending on the construct used.

All constructs described here were also used for Nicotiana tabaccum Agrobacterum-mediated leaf disc (Horsh et al., 1985, Science, 227, 1229-1231) and Brassica napus (cv. Westar) hypocotyl (Radke et al., 1988, Theor. Appl. Genet., 75, 685-694) transformations. Despite a 10-20 fold difference in genome size of Arabidopsis compared to Brassica napus and tobacco, respectively, and higher density of transcribed regions in Arabidopsis compared to tobacco and Brassica, the frequency of transformants of Brassica and tobacco obtained with translational fusion vectors, was comparable to that of Arabidopsis (15-25 times lower compared to transcriptional vectors).

Appendix

Seed Sterilization

Soak the seeds in 1% PPM solution for at least 2 hours (overnight is preferable). Wash the seeds in 70% EtOH for 1 minute than sterilize in 10% chlorine solution with 0.01% SDS or Tween 20) in a 250 ml flask placed on the rotary shaker. Wash the seeds in 0.5 L of sterile water.

| TVL | |
|---|---|
| 0.3 M sorbitol | |
| 0.05 M CaCl₂ × 2H₂O | |
| pH 5.6-5.8 | |
| W5 | |
| 18.4 g/L CaCl₂ × 2H₂O | |
| 9.0 g/L NaCl | |
| 1.0 g/L glucose | |
| 0.8 g/L KCl | |
| pH 5.6-5.8 | |
| CIM | |
| Macro MS | |
| Micro MS | |
| Vitamin B5 | |
| MES | 500 mg/L |
| PVP | 500 mg/L |
| Sucrose | 30 g/L |
| 2.4-D | 5 mg/L |
| Kin | 0.25 mg/L |
| Gelrite | 3 g/L |
| pH | 5.6-5.8 |
| Greening Medium (GM) | |
| Macro MS | |
| Micro MS | |
| Vit B5 | |
| MES | 500 mg/L |
| PVP | 500 mg/L |
| Sucrose | 30 g/L |
| BA | 2 mg/L |
| Kin | 0.5 mg/L |
| NAA | 0.1 mg/L |
| pH | 5.6-5.8 |
| Regeneration Medium | |
| Macro MS | |
| Micro MS | |
| Vit B5 | |
| MES | 500 mg/L |
| PVP | 500 mg/L |
| Sucrose | 30 g/L |
| ABA | 1 mg/L |
| BA | 0.5 mg/L |
| IAA | 0.1 mg/L |
| pH | 5.6-5.8 |
| Enzyme solution | |
| 1% cellulase R10 | |
| 0.2% macerase R10 | |
| 0.1% dricelase | |
| dissolved in 8 pM macrosalt with 0.5 M | |
| pH 5.6-5.8 | |
| PEG solution | |
| 40% (w/v) of PEG-2000 in H₂O | |
| MIM | |
| Macro MS | |
| Micro MS | |
| Vitamin B5 | |
| MES | 500 mg/L |
| PVP | 500 mg/L |
| Sucrose | 30 g/L |
| ABA | 1 mg/L |
| BA | 0.5 mg/L |
| IAA | 0.1 mg/L |
| Gelrite | 3 g/L |
| pH | 5.6-5.8 |
| High Auxine Medium (HAM) | |
| Macro MS | |
| Micro MS | |
| Vit B5 | |
| MES | 500 mg/L |
| PVP | 500 mg/L |
| Sucrose | 30 g/L |
| NAA | 5 mg/L |
| Kin | 0.25 mg/L |
| BA | 0.25 mg/L |
| pH | 5.6-5.8 |

Hormone solutions were filter sterilized and added to the autoclaved media.

1. A process for producing transgenic plants or plant cells, comprising expressing a coding sequence of interest under transcriptional and translational control of host nuclear transcriptional and translational elements by:
(i) introducing into the nuclear genome of host plants or plant cells for said transgenic plants or plant cells a vector comprising said coding sequence of interest which is devoid of
(a) an upstream element of initiation of transcription functional in the host plants or plant cells operably linked to said coding sequence of interest and required for its transcription;
(b) an upstream element of initiation of translation functional in the host plants or plant cells and operably linked to said coding sequence of interest;
wherein said coding sequence of interest codes for a selectable marker conferring a selection advantage; and
(ii) subsequently selecting plant cells or plants expressing said selectable marker, whereby the selection advantage conferred by said selectable marker is used.

2. The process according to claim 1, wherein said vector further comprises splicing donor and/or acceptor site(s) upstream and/or downstream of said coding sequence of interest.

3. The process according to one of claims 1 or 2 claim 1, wherein said vector further comprises one or more cistrons downstream of said coding sequence of interest, said cistrons being joined to said coding sequence of interest.

4. The process according to claim 3, wherein at least one of said one or more cistrons downstream of said coding sequence of interest is operably linked to transcriptional and/or translational element(s) located downstream of said coding sequence of interest.

5. The process according to claim 1, wherein said vector further contains one or more sequences coding for targeting signal peptides operably linked to said coding sequence of interest or said cistron(s).

6. The process according to claim 1, wherein said vector further contains one or more sequence(s) encoding proteolytic cleavage sites next to or within said coding sequence of interest or said cistron(s).

7. The process according to claim 6, wherein said sequence(s) encoding proteolytic cleavage sites next to or within said coding sequence of interest or said cistron(s) are autocatalytic.

8. The process according claim 1, wherein said transgenic plants or plant cells are genetically modified or transfected so as to provide site-specific proteases necessary for cleavage of expressed fusion proteins.

9. The process according to claim 1, wherein said vector further contains one or more transcriptional enhancers operably linked to said coding sequence of interest or said cistron(s).

10. The process according to claim 1, wherein said vector further contains one or more translational enhancer(s) operably linked to said coding sequence of interest or said cistron(s).

11. The process according to claim 1, wherein said vector further contains one or more recombination sites recognized by site-specific recombinases.

12. The process according to claim 1, wherein hybrid messenger RNA is produced comprising RNA derived from nuclear DNA of said transgenic plants or plant cells and RNA derived from said coding sequence of interest.

13. The process according to claim 12, wherein said hybrid messenger RNA encodes multiple heterologous polypeptide sequences.

14. The process according to claim 12, wherein said hybrid messenger RNA is at least partially complementary to a messenger RNA present in said transgenic plants or plant cells.

15. The process according to claim 12, wherein translation of said hybrid messenger RNA leads to a fusion protein.

16. The process according to claim 15, wherein said fusion protein comprises multiple heterologous polypeptide sequences.

17. The process according to claim 1, wherein said coding sequence of interest is of plant origin.

18. The process according to claim 1, wherein said vector contains functional elements of plant origin only.

19. The process according to claim 1, wherein said coding sequence of interest is further devoid of an element of termination of transcription functional in the host plants or plant cells and operably linked to said coding sequence of interest.

20. The process according to claim 1, wherein expression of said coding sequence of interest results in polypeptide formation.

21. (canceled)

22. RNA obtained by using the process according to claim 1.

23. Protein or polypeptide obtained by using the process according to claim 1.

24. Plant cells, plants and their progeny obtained by the process according to claim 1.

25. (canceled)

26. (canceled)

27. The plant cells, plants and their progeny according to claim 24, characterized by containing in the nuclear genome a coding sequence of a selectable marker under transcriptional and translational control of host nuclear transcriptional and translational elements, said coding sequence being devoid of
(a) an upstream element of initiation of transcription functional in the host plants or plant cells operably linked to said coding sequence of interest and required for its transcription; and
(b) an upstream element of initiation of translation functional in the host plants or plant cells and operably linked to said coding sequence of interest.

28. A method of targeted transformation of plant cells or plants, wherein the plant cells or plants according to claim 27 are used.
